(19) United States
(12) Patent Application Publication
(10) Pub. No.: US 2011/0028526 A1
(43) Pub. Date: Feb. 3, 2011

(54) VALSARTAN SOLID ORAL DOSAGE FORMS AND METHODS OF MAKING SUCH FORMULATIONS

(76) Inventors: Amol Matharu, Bedminster, NJ (US); Agnes Taillardat, Seewen So (CH); Robert F. Wagner, Hillsborough, NJ (US)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/919,165

(22) PCT Filed: Feb. 27, 2009
(86) PCT No.: PCT/US09/35368

§ 371 Date: Aug. 24, 2010

Related U.S. Application Data
(60) Provisional application No. 61/032,187, filed on Feb. 28, 2008.

(51) Int. Cl. A61K 31/41; A61P 9/04

(52) U.S. Cl. 514/381

(57) ABSTRACT

The invention relates to melt granulation processes for preparing immediate release and sustained release pharmaceutical formulations comprising valsartan.

### FIELD OF THE INVENTION

The present invention relates to a melt granulation process for preparing a pharmaceutical tablet formulation of valsartan formulated

### BACKGROUND

Methods of manufacturing valsartan and the resulting formulations have included dry compaction as disclosed in U.S. Pat. No. 6,294,197 to Novartis and WO 2007/052307 to Lupin The manufacturing methods used Valsartan (and its salts or hydrate forms) have ranged from dry compaction (U.S. Pat. No. 6,294,197, Novartis) (WO2007052307, Lupin), dry blending (WO 2005041941, Zentiva) to wet granulation (WO 2006066961, Krka). The wet granulation methods have used conventional wetting techniques such as pot granulation using aqueous or non-aqueous solvents. Fluid bed granulation has also been taught by EP1674080, Krka. These methods have been used to obtain combination products as well, and have primarily resulted in an immediate release product.

Valsartan is commercially sold under the trademark Diovan® and is the largest selling anti-hypertension medication in its class in the world.

Traditionally, melt methods have been used for MR formulations using waxes and equipment such as jacketed vessels and extruders at elevated temperatures. In a pharmaceutical context, extruders have been used for the manufacture of solid dispersion and/or solid solutions that have required at least a partial melting of the therapeutic compound. It has been found that the use of melt methods can be useful in the preparation of IR melt granulated solid dosage forms without the need for the melting of the therapeutic compound. The methods used for manufacturing modified release (MR) immediate release (IR) formulations containing fine particle size of valsartan as a starting material typically require methodology that required multiple steps—for example, roller compaction and wet granulation. The melt method is a dry form of agglomeration process that is explored for an MR and IR product in this invention

Thus, there is a need for additional methods of preparing valsartan that has the ability to produce both IR and MR formulations of valsartan by using a quick, robust and dry process that reduces processing time and improves stability. This invention addresses that need by utilizing melt granulation techniques. A particularly inventive aspect of the present invention is the use of jacketed vessels or melt extruders to provide for melt granulation compounding.

### SUMMARY OF THE INVENTION

In a first embodiment the present invention features a process for making an IR pharmaceutical composition comprising valsartan or pharmaceutically acceptable salts thereof that comprises mixing valsartan or pharmaceutically acceptable salts thereof with a diluent to form a pre-mix; melting wax in a pre-heated jacket vessel; adding the pre-mix to the jacketed vessel containing the melted wax to obtain a uniform granulation; cooling the granulation by removing it from the jacketed vessel; sizing the cooled granulation; mixing a lubricant into the cooled granulation and compressing into a solid oral dosage form, e.g. tablets. In a particular aspect, a disintegrant is added to the pre-mix.

In a second embodiment the present invention is directed to IR solid oral dosage forms made according to the process of the first embodiment of the invention.

In a third embodiment, the present invention is directed to a process for making an MR pharmaceutical composition comprising valsartan or pharmaceutically acceptable salts thereof comprising (a) preparing a blend by combining valsartan or pharmaceutically acceptable salts thereof, a hydrophilic erodible component and a hydrophobic component as a monolithic or single phase homogenous system; and (b) compressing the blend into a solid oral dosage form, e.g. a tablet. The MR forms are devoid of agents that swell rapidly or disintegrate.

In a fourth embodiment the present invention is directed to MR solid oral dosage forms made according to the process of the third embodiment of the invention.

### DETAILED DESCRIPTION

The present invention relates to processes for preparing IR and MR pharmaceutical compositions of valsartan or pharmaceutically acceptable salts thereof and the dosage forms prepared by these processes. The inventive process features a melt granulation formulation of valsartan or pharmaceutically acceptable salts thereof, prepared through the use of a jacketed vessel or a melt extruder.

Valsartan or ((S)-N-valeryl-N-{[2′-(1H-tetrazole-5-yl)-biphenyl-4-yl]-methyl}-valine) suitable for use in the present invention can be purchased from commercial sources or can be prepared according to known methods. For example, the preparation of Valsartan is described in U.S. Pat. No. 5,399,578, the entire disclosure of which is incorporated by reference herein. Valsartan may be used for purposes of this invention in its free form as well as in any suitable salt form.

Also included within the scope of the present invention are the salts, esters, amides, prodrugs, active metabolites, analogs, and the like of Valsartan, particularly the calcium salt of Valsartan. A detailed description of the calcium salt and process of making are disclosed in published U.S. Patent Application No. 2003/0207930, the contents of which are fully incorporated by reference herein in their entirety.

As used herein the term “pharmaceutical composition” means a mixture containing a valsartan or pharmaceutically acceptable salts thereof to be administered to a mammal, e.g., a human in order to prevent, treat or control a particular disease or condition affecting the mammal.

As used herein the term “pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein, the term “immediate release” refers to the rapid release of the majority of the therapeutic compound, e.g., greater than about 50%, about 60%, about 70%, about 80%, or about 90% within a relatively short time, e.g., within 1 hour, 40 minutes, 30 minutes or 20 minutes after oral ingestion. Particularly useful conditions for immediate-release are release of at least or equal to about 80% of the therapeutic compound within thirty minutes after oral ingestion. The particular immediate release conditions for a specific therapeutic compound will be recognized or known by one of ordinary skill in the art.

As used herein, the term “modified release”, refers to the gradual but continuous or sustained release over a relatively extended period of the therapeutic compound content after oral ingestion. The release will continue over a period of time and may continue through until and after the pharmaceutical composition reaches the intestine. Sustained release may also refer to delayed release in which release of the therapeutic compound does not start immediately when the pharmaceutical composition reaches the stomach but is delayed for a period of time, for instance, until when the pharmaceutical composition reaches the intestine when the increasing pH is used to trigger release of the therapeutic compound from the pharmaceutical composition.

The pharmaceutical compositions of the present invention may be prepared by the vessel method disclosed in WO 03/004009 (Geneva) or by melt extruders as described in WO 06/122021 (Novartis), both of which are hereby incorporated by reference in their entirety as if set forth in full herein.

In general, an extruder includes a rotating screw(s) within a stationary barrel with an optional die located at one end of the barrel. Along the entire length of the screw, distributive kneading of the materials (e.g., the therapeutic compound, release retarding material, and any other needed excipients) is provided by the rotation of the screw(s) within the barrel. Conceptually, the extruder can be divided into at least three sections: a feeding section; a heating section and a metering section. In the feeding section, the raw materials are fed into the extruder, e.g. from a hopper. In the heating section, the raw materials are heated to a temperature less than the melting temperature of the poorly compressible therapeutic compound. After the heating section is a metering section in which the mixed materials are extruded through an optional die into a particular shape, e.g., granules or noodles. Types of extruders particularly useful in the present invention are single-, twin- and multi-screw extruders, optionally configured with kneading paddles.

Once the granules are obtained, the granules may be formulated into oral forms, e.g., solid oral dosage forms, such as tablets, pills, lozenges, caplets, capsules or sachets, by adding additional conventional excipients which comprise an external phase of the pharmaceutical composition. The external phase of the pharmaceutical composition can also comprise an additional therapeutic compound. Such solid oral dosage forms, e.g., are unit oral dosage forms. Examples of such excipients include, but are not limited to, release retardants, plasticizers, disintegrants, binders, lubricants, glidants, stabilizers, fillers and diluents. One of ordinary skill in the art may select one or more of the aforementioned excipients with respect to the particular desired properties of the solid oral dosage form by routine experimentation and without any undue burden. The amount of each excipient used may vary within ranges conventional in the art. The following references which are all hereby incorporated by reference discloses techniques and excipients used to formulate oral dosage forms. See The Handbook of Pharmaceutical Excipients, 4<th> edition, Rowe et al., Eds., American Pharmaceuticals Association (2003); and Remington: the Science and Practice of Pharmacy, 20<th> edition, Gennaro, Ed., Lippincott Williams & Wilkins (2003).

One aspect of the present invention relates to a process for preparing an IR pharmaceutical formulation of valsartan, which comprises mixing valsartan or pharmaceutically acceptable salts thereof with a diluent to form a pre-mix; melting wax in a pre-heated jacket vessel; adding the pre-mix to the jacketed vessel containing the melted wax to obtain a uniform granulation; cooling the granulation by removing it from the jacketed vessel; sizing the cooled granulation; mixing a lubricant into the cooled granulation and compressing into a solid oral dosage form, e.g. tablets. In a particular aspect, a disintegrant is added to the pre-mix

Valsartan can be present in a range of from about 5 to about 90% of the pharmaceutical inventions, preferably from about 5 to about 55%, more preferably from about 5 to about 45% and most preferably from about 10 to about 45%.

Examples of waxes suitable in the above described IR formulation include, but are not limited to, behenic acid, glyceryl monostearate, glyceryl palmitostearate, glyceryl behenate, carnauba wax, beeswax and spermaceti wax. Examples of hydrocarbons include, but are not limited to, microcrystalline wax and paraffin. Examples of fatty alcohols, i.e., higher molecular weight nonvolatile alcohols that have from about 14 to about 31 carbon atoms include, but are not limited to, cetyl alcohol, e.g., CRODACOL C-70 from Croda Corp. (Edison, N.J.); stearyl alcohol, e.g., CRODACOL S-95 from Croda Corp; lauryl alcohol; and myristyl alcohol. Examples of fatty acids which may have from about 10 to about 22 carbon atoms include, but are not limited to, stearic acid, e.g., HYSTRENE 5016 from Crompton Corp. (Middlebury, Conn.); decanoic acid; palmitic acid; lauric acid; and myristic acid. Cetyl alcohol and stearyl alcohol are preferred. The waxes may be present in an amount from about 5% to about 75% by weight of the composition, preferably from about 5% to about 50% and even more preferably from about 10% to about 35% by weight of composition and most preferably from about 20 to about 35% by weight of the composition.

The diluents which may be used include, but are not limited to, microcrystalline cellulose (MCC), lactose, mannitol, calcium phosphate or any pharmaceutically acceptable. The choice is made based on the dissolution characteristics that are needed (e.g. IR or MR). The diluents may be present in an amount from about 5% to about 75% by weight of the composition, preferably from about 10% to about 60% by weight of composition.

The disintegrants can be extragranular or intragranular or both. Examples of pharmaceutically acceptable disintegrants include, but are not limited to, starches; clays; celluloses, such as MCC; alginates; gums; cross-linked polymers, e.g., cross-linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. The disintegrant may be present in an amount from about 0% to about 20% by weight of the composition, preferably from about 0.1% to about 20% by weight of composition.

The glidants which may be used include but are not limited to talcs or colloidal silicon dioxide. The glidants may be present in an amount from about 0.1% to about 5% by weight of the composition, preferably the glidant is present in an amount of from about 0.1% to about 3% by weight of composition.

The lubricants include but are not limited to magnesium, calcium, sodium stearate and sodium stearyl fumarate. The lubricants may be present in an amount from about 0.1% to about 5% by weight of the composition, preferably in an amount of from about 0.1% to about 3% by weight of composition.

For the MR tablet, the preferred hydrophobic components are waxes of natural or synthetic origin e.g. Bees was, carnauba wax, cetyl alcohol and stearyl alcohol. Optionally, other release controlling agents such as celluloses (HPMC, EC, or HPC), but not limited to celluloses, may be combined with the waxes.

An IR formulation according to the invention comprises between about 5% to about 55% valsartan or pharmaceutically acceptable salts thereof, preferably between about 5% and about 35% and even more preferably between about 10% and about 45%; between about 20% to about 35% wax; between about 25% to about 75% diluent, preferably between about 10 and about 60%; between about 0% to about 20% disintegrant, preferably between about 0.1% and about 20% and even more preferably between about 5% and about 20%; between about 0% to about 5% glidant, preferably between about 0.1% and about 3% and between about 0.1% to about 5% lubricant, preferably between about 0.1 and about 3%.

In another embodiment, the present invention is directed to a process for making an MR pharmaceutical composition comprising valsartan or pharmaceutically acceptable salts thereof comprising (a) preparing a blend by combining valsartan or pharmaceutically acceptable salts thereof, a hydrophilic erodible component and a hydrophobic component as a monolithic or single phase homogenous system; and (b) compressing the blend into a solid oral dosage form, e.g. a tablet. The MR forms are devoid of agents that swell rapidly or disintegrate.

The blend may optionally comprise other pharmaceutical acceptable excipients and the blend may optionally be lubricated prior to being compressed into tablets.

The hydrophilic erodible component of the present invention is a pharmaceutical acceptable excipient which is a water-loving soluble/gellable agent. These components possess properties, such as the ability to imbibe external fluid and dissolve/erode over a period of time. Typical hydrophilic erodible components include hydroxypropylmethyl cellulose; soluble fillers, such as lactose; tablet disintegrants, such ascroscarmellose sodium; binders, such as polyvinylpyrrolidone; gums, such as guar and xanthan gums.

Examples of watersoluble and/or swellable hydrophilic polymers include solid polyethylene glycol with molecular weights greater than 400 (MW>400), celluloses (hydroxymethyl cellulose, hydroxypropyl cellulose, hydroxyethyl cellulose, carboxymethyl cellulose, carboxyethyl cellulose, sodium carboxymethyl cellulose, sodium alginate, methyl cellulose, hydroxypropyl methyl cellulose), carboxypolymethylene, gums (acacia gum, guar gum, tragencanth gum and xanthan gum), polyethylene oxide and the like. High molecular weight cellulose derivatives are preferred as the hydrophilic erodible component.

The hydrophobic component is a pharmaceutical acceptable excipient which is water insoluble and does not dissolve in water over a period of time. Typical hydrophobic components include ethyl cellulose, methacrylic acid polymers and copolymers, such as

EUDRAGIT NE 30 D from Rohm and Haas, fatty acids and esters thereof, such as stearic acid, behenic acid, glyceryl monostearate, glyceryl palmitostearate, glyceryl behenate and other waxes, such as carbuna wax. Also included are high molecular weight fatty alcohols, such as cetyl alcohol and the like. Cetyl alcohol and stearyl alcohol are preferred as the hydrophobic component.

Valsartan can be present in a range of from about 0.1 to about 99% of the pharmaceutical inventions, preferably from about 5 to about 55%, more preferably from about 5 to about 45% and most preferably from about 10 to about 45%.

The hydrophilic erodible component typically represents from about 10% to about 90% by weight of the formulation. Preferably, the hydrophilic erodible component is present in the formulation in amount of from about 30% to about 70% by weight.

The hydrophobic component typically represents from about 1% to about 30% by weight of the formulation. Preferably, the hydrophobic component is present in the formulation in amount of from about 15% to about 25% by weight.

Typically, the ratio of hydrophilic erodible component to hydrophobic component is in the range from about 9:1 to 1:1. Preferably the ratio is in the range from about 2:1 to 3:1.

Preferred formulations comprise from about 40% to about 60% by weight of valsartan and comprise the hydrophilic erodible component and hydrophobic component in a ratio of from about 2:1 to 3:1.

Valsartan, hydrophilic erodible component and hydrophobic component are blended by standard techniques. Typically, the components are added to a standard blending apparatus and blended. Hydrophobic components which are solid at room temperature, such as waxes, are often liquefied before and/or during the blending operation.

In another embodiment, valsartan and hydrophilic erodible component are pre-mixed by standard techniques and then combined with the hydrophobic component. The pre-mixed components are combined with the hydrophobic component by a variety of techniques, such as adding the hydrophobic component to the blending apparatus containing the pre-mixed components. The fluidized bed technique may also be used and is especially appropriate when the hydrophobic component is ethyl cellulose or a polymethacrylic acid polymer or co-polymer.

The blend produced by combining valsartan, hydrophilic erodible component and hydrophobic component is typically a monolithic or single phase homogenous free flowing powder. As is typical when formulating tablets, the free flowing powder blend is often milled or sieved in order to control the particle size of the blend and to remove large agglomerates.

If needed, the blend my optionally be lubricated prior to compression into tablets.

Typical lubricants include magnesium stearate and stearic acid. However, the presence of the hydrophobic component often renders additional lubrication unnecessary. Additional lubricants will generally represent 0% to about 6% by weight of the tablet formulation.

In addition to the poorly-compressible pharmaceutical agent, hydrophilic erodible component, hydrophobic component and optional lubricant, the formulations of the present invention may contain additional pharmaceutical excipients, such as flavoring agents, binders and/or fillers.

It is preferred for the process to be carried out under substantially anhydrous conditions.

The release rate of valsartan is controlled by the hydrophilic erodible agent and hydrophobic agent. Thus, an immediate-release formulation will typically contain the hydrophilic erodible component and hydrophobic components in a ratio of from about 1:9 to 2:8. Increasing the amount of hydrophilic erodible component will extend the release rate of valsartan. Thus, sustained-release dosage forms typically contain the hydrophilic erodible component and hydrophobic components in a ratio of from about 3:1 to 2:1.

The dissolution profile obtained in phosphate buffer (pH 6.8), USP Apparatus II, for tablets prepared according to the present invention are:

| Time (hrs) | % Dissolved |
|---|---|
| 1 | 10-50 |
| 4 | 30-80 |
| 7 | 40-100 |

The invention is applicable to sublingual lozenges, buccal tablets, oral lozenges, suppositories and compressed tablets, the latter being intended to be swallowed in unit dosage form and which upon ingestion according to a prescribed regimen give slow and regular release of valsartan of a fixed percentage in the intestinal tract.

In a preferred embodiment, the hydrophilic erodible component is hydroxypropyl methylcellulose and the hydrophobic component is stearyl alcohol, wherein the hydrophilic agent and hydrophobic agent are in a ratio of from about 3:1 to about 2:1.

Most preferably, a tablet comprises from about 25-75% by weight of valsartan, a hydrophilic erodible component which is hydroxypropyl methylcellulose and a hydrophobic component which is stearyl alcohol and a weight ratio of hydrophilic erodible component to hydrophobic component in the range from 3:1 to about 2:1.

In one embodiment, the tablet may comprise from about 40 to about 60% by weight valsartan. The pharmaceutical compositions of the present invention may be employed to treat hypertension, congestive heart failure, angina, myocardial infarction, arteriosclerosis, diabetic nephropathy, diabetic cardiac myopathy, renal insufficiency, peripheral vascular disease, stroke, left ventricular hypertrophy, cognitive dysfunction, headache, or chronic heart failure by administering a therapeutically effective amount of the pharmaceutical compositions of the present invention to a subject in need of such treatment.

The following examples are illustrative, but do not serve to limit the scope of the invention described herein. The examples are meant only to suggest a method of practicing the present invention.

### Example 1

| | | | | Amount |
|---|---|---|---|---|
| Item no. | Ingredient | % | mg/unit | per batch |
| 1 | Valsartan | 48.54 | 500 | 55 kg |
| | Hydroxypropyl methylcellulose | 31.06 | 320 | 39.05 kg |
| 3 | Stearyl alcohol | 19.41 | 200 | 18.15 kg |
| 4 | Magnesium stearate | 0.97 | 10 | 1.1 kg |
| Total | | 100 | 1030 | 113.3 kg |

Valsartan is first de-lumped using Fit-mill equipped with 0.050″ screen at medium speed. De-lumped valsartan and hydroxypropyl methyl cellulose (available as Methocel K100 M Premium CR, Dow Chemical Company, MI) are mixed in a 340 Qt. AMF

Planetary Mixer and mixed for 10 minutes to form a pre-mix blend. The pre-mix blend is transferred to drums. To a pre-heated jacketed bowl of 340 Qt. AMF Planetary Mixer, stearyl alcohol is added and allowed to melt to form a clear liquid at the jacket temperature of not less than 65 C. To the melted wax, pre-mix is added and mixed until a uniform granulation is obtained while heating at the jacket temperature of not less than 65 C. The granulation is transferred to trays lined with Kraft paper and cooled down to a temperature of 25 C-30 C. The cooled granulation is sized using a low energy screening/milling device such as a Glatt Quick sieve equipped with 1.5 mM screen. The lubrication is performed using magnesium stearate in a 30 cu. ft. Gemco Blender. The final-mix obtained is compressed into tablets using Manesty Unipress Diamond using modified oval tools. The hardness of the tablets obtained was 10-18 SCU.

The dissolution profile (average, n=6) obtained in phosphate buffer (pH 6.8), USP Apparatus II, is:

| Time (hours) | % dissolved |
|---|---|
| 1 | 31.7 |
| 4 | 64.2 |
| 7 | 80.1 |

### Example 2

| | Ingredient | | | Amount |
|---|---|---|---|---|
| Item no. | Valsartan I with | % | mg/unit | per batch |
| 1 | magnesium stearate 0.5% | 48.8 | 502.5 | 40.0 kg |
| 2 | Hydroxypropyl methylcellulose | 28.8 | 297 | 23.76 kg |
| 3 | Stearyl alcohol | 21.4 | 220 | 17.6 kg |
| 4 | Colloidal silicon dioxide | 0.3 | 3 | 0.24 kg |
| 5 | Magnesium stearate | 0.7 | 7.5 | 0.6 kg |
| TOTAL | | 100 | 1030 | 82.4 kg |

Valsartan with 0.5% magnesium stearate and hydroxypropyl methyl cellulose (available as Methocel K100 M Premium CR, Dow Chemical Company, MI) are mixed in a

PMA 300 Fielder High Shear to form a pre-mix blend. The pre-mix blend is transferred to drums. To a pre-heated jacketed bowl of 340 Qt. AMF Planetary Mixer, stearyl alcohol is added and allowed to melt to form a clear liquid at the jacket temperature of not less than 65 C. To the melted wax, pre-mix is added and mixed until a uniform granulation is obtained while heating at the jacket temperature of not less than 65 C. The granulation is transferred to trays lined with Kraft paper and cooled down to a temperature of 25 C-30 C. The cooled granulation is sized using a low energy screening/milling device such as a Quadro Co-Mill equipped with 93 screen. The Pre-lubrication and Lubrication is performed using colloidal silicon dioxide and magnesium stearate, respectively, in a Patterson-Kelley Blender. The final-mix obtained is compressed into tablets using Manesty Unipress Diamond using modified oval tools. The hardness of the tablets obtained was 10-18 SCU. The dissolution profile of the tablets matched that of Glucophagee XR 500 mg (Bristol-Myers Squibb, N.J.).

The dissolution profile (average, n=6) obtained in phosphate buffer (pH 6.8), USP Apparatus II, is:

| Time (hours) | % dissolved |
|---|---|
| 1 | 31.5 |
| 4 | 63.5 |
| 7 | 80.9 |

### Example 3

### Immediate-Release Formulation

| Item no. | Ingredient | % | mg/unit |
|---|---|---|---|
| 1 | Valsartan | 71.4 | 500 |
| 2 | Hydroxypropyl methylcellulose | 10.6 | 74 |
| 3 | Stearyl alcohol | 17.0 | 119 |
| 4 | Colloidal silicon dioxide | 0.2 | 1.4 |
| 5 | Magnesium stearate | 0.8 | 5.6 |
| TOTAL | | 100 | 700 |

Valsartan and hydroxypropyl methyl cellulose (available as Pharmacoat 606,

Shin-Etsu Chemical Co. Ltd., Japan) are mixed in a 500 mL glass beaker with the help of a stainless steel spatula. Stearyl alcohol is melted in a glass beaker. To the melted wax, premix is added and mixed until a uniform granulation is obtained while heating at temperature of not less than 65 C. The granulation is transferred to Kraft paper and cooled down to a temperature of 25 C-30 C. The cooled granulation is sized using screen# 20. The Prelubrication and Lubrication is performed using colloidal silicon dioxide and magnesium stearate, respectively, in a glass beaker using a stainless steel spatula. The final-mix obtained is compressed into tablets using Carver hydraulic press. The hardness of the tablets obtained was 8 SCU.

The dissolution profile (average, n=3) obtained in phosphate buffer (pH 6.8), USP Apparatus II, is:

| Time (minutes) | % dissolved |
|---|---|
| 15 | 43.9 |
| 30 | 76.1 |
| 45 | 97.1 |
| 60 | 100.2 |

### Example 4

### Immediate-Release Formulation

| Item no. | Ingredient | % | mg/unit |
|---|---|---|---|
| | Valsartan | 50.0 | 500 |
| 2 | Microcrystalline cellulose | 32.0 | 320 |
| 3 | Stearyl alcohol | 17.0 | 170 |
| 4 | Colloidal silicon dioxide | 0.2 | 2 |
| 5 | Magnesium stearate | 0.8 | 8 |
| TOTAL | | 100 | 1000 |

Valsartan and microcrystalline cellulose are mixed in a 500 mL glass beaker with the help of a stainless steel spatula. Stearyl alcohol is melted in a glass beaker. To the melted wax, pre-mix is added and mixed until a uniform granulation is obtained while heating at temperature of not less than 65 C. The granulation is transferred to Kraft paper and cooled down to a temperature of 25 C-30 C. The cooled granulation is sized using screen

No. 20. The pre-lubrication and lubrication is performed using colloidal silicon dioxide and magnesium stearate, respectively, in a glass beaker using a stainless steel spatula. The final-mix obtained is compressed into tablets using Carver hydraulic press. The hardness of the tablets obtained was 8 SCU.

The dissolution profile (average, n=3) obtained in phosphate buffer (pH 6.8), USP Apparatus II, is:

| Time (minutes) | % dissolved |
|---|---|
| 5 | 75.6 |
| 15 | 100.4 |

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

1. A process for preparing a MR pharmaceutical formulation of valsartan, which comprises the steps of (a) preparing a blend by combining valsartan, a hydrophobic component and, optionally, an hydrophilic component; and (b) compressing the blend into a solid oral dosage form tablet.

2. The process of claim 1 wherein the solid oral dosage form is a tablet.

3. A process according to claim 1 further comprising mixing an optional lubricant with the blend prior to compressing the blend into a tablet.

4. A process according to claim 1, wherein the process is carried out under substantially anhydrous conditions.

5. A process according to claim 1, wherein the hydrophilic erodible component is selected from the group consisting of hydroxypropyl methylcellulose, lactose, croscarmellose sodium, polyvinylpyrrolidone, guar and xanthan gums, polyethylene glycol (MW>400), celluloses, hydroxymethyl cellulose, hydroxypropyl cellulose, hydroxyethyl cellulose, carboxymethyl cellulose, carboxyethyl cellulose, sodium carboxymethyl cellulose, sodium alginate, methyl cellulose, carboxypolymethylene, acacia gum, tragencanth gum and polyethylene oxide.

6. A process according to claim 7, wherein the hydrophilic erodible component is hydroxypropyl methylcellulose.

7. A process according to claim 1, wherein the hydrophobic component is selected from the group consisting of ethyl cellulose, methacrylic acid polymers and copolymers, fatty acids and esters thereof, waxes and high molecular weight fatty alcohols.

8. A process according to claim 1, wherein the hydrophobic component is selected from the group consisting of EUDRAGIT NE 30 D from Rohm and Haas, stearic acid, behenic acid, glyceryl monostearate, glyceryl palmitostearate, glyceryl behenate, carbuna wax and cetyl alcohol.

9. A process according to claim 8, wherein the hydrophobic component is selected from the group consisting of cetyl alcohol and stearyl alcohol.

10. A process according to claim 1, wherein the valsartan comprises from about 0.1% to about 99% by weight of the formulation.

11. A process according to claim 1, wherein the hydrophilic erodible component comprises from about 10% to about 90% by weight of the formulation.

12. A process according to claim 1, wherein the hydrophobic component comprises from about 10% to about 30% by weight of the formulation.

13. A process according to claim 1, wherein the ratio of hydrophilic erodible component to hydrophobic component is 9:1 to 1:1.

14. (canceled)

15. (canceled)

16. A process for preparing an immediate-release pharmaceutical formulation of valsartan, which comprises the steps of: (a) mixing valsartan with a diluent to form a pre-mix; (b) melting wax in a pre-heated jacket vessel and adding the pre-mix to the vessel to obtain uniform granulation; (c) cooling the granulation; and (d) compressing the blend into a solid oral dosage form.

17. The process of claim 16 wherein the solid oral dosage form is a tablet.

18-23. (canceled)
